(19) United States
(12) Patent Application Publication
(10) Pub. No.: US 2006/0135940 A1
(43) Pub. Date: Jun. 22, 2006

(54) Programmed pulsed infusion methods and devices

(75) Inventor: Shailendra Joshi, Edgewater, NJ (US)

Correspondence Address: BROWN RAYSMAN MILLSTEIN FELDER & STEINER LLP, 900 THIRD AVENUE, NEW YORK, NY, 10022, US

(73) Assignee: THE TRUSTEES OF COLUMBIA, New York, NY (US)

(21) Appl. No.: 11/182,703

(22) Filed: Jul. 14, 2005

Related U.S. Application Data
(63) Continuation-in-part of application No. 10/337,677, filed on Jan. 6, 2003.
(63) Continuation-in-part of application No. 10/879,850, filed on Jun. 28, 2004.

(51) Int. Cl. A61M 31/00

(52) U.S. Cl. 604/500; 604/890.1; 700/1

(57) ABSTRACT

This invention disclosed herein provides methods, devices, software products, and systems for infusing a fluid into a blood vessel is provided that includes the step of administering the fluid into the blood vessel in programmed pulses. The programmed pulses generally defined by programmed pulse variables that include a fluid flow rate, a frequency, and a duration. Values of the programmed pulse variables may be determined based at least in part on a fluid property of the fluid to be infused that is relevant to streaming, blood flow in the blood vessel to be infused, a catheter size, or a patient profile.

### CROSS-REFERENCE TO RELATED APPLICATIONS

The present application is a continuation-in-part of U.S. patent application Ser. No. 10/337,677, filed Jan. 6, 2003 and a continuation-in-part of U.S. patent application Ser. No. 10/879,850, filed Jun. 28, 2004, both of which are hereby incorporated by reference thereto.

### BACKGROUND OF THE INVENTION

The invention generally relates to infusion techniques. Specifically, the invention discussed herein relates to methods and devices that generate more predictable drug concentrations downstream by introducing or administering a drug dissolved in fluid into a blood vessel in a manner to compensate for or otherwise overcome streaming and variable velocity related to tissue uptake.

In certain blood vessels, such as the carotid artery, the velocity or flow of the blood therein is such that fluid introduced into the blood vessel, particularly in small quantities, has a tendency to follow the stream of the blood flow and pass a site targeted for the fluid. Streaming is exhibited, particularly in laminar flow fluid patterns due to the variable velocity pattern occurring with a flowing fluid. Streaming may be explained with reference to FIG. 1, which shows a section of a blood vessel 102 with a laminar fluid flow along the X-axis. The velocity of the fluid varies as shown by the fluid velocity (V) profile 104, which is a function of the distance (D) along the Y-axis. A small quantity of fluid 108 introduced into the flow has a tendency to follow the direction of the higher velocity streamlines 104 and pass a possible target 112 for the fluid, rather than in the direction of the lower velocity streamline 110 to the target 112. Because of streaming, delivering fluids, such as drugs, in small quantities, particularly via the carotid artery, has proven to be unreliable and the effects of the drugs consequently not consistent.

One attempt to overcome streaming has been to infuse drugs by periodically pulsing the fluid at high infusion rates and phased with diastole, in an attempt to introduce the fluid into the carotid artery at a sufficient rate to overcome the streaming in at the time the blood flow at the injection site is at the lowest. This method of overcoming streaming, however, has numerous shortcomings. Although low average infusion rates are achieved, the fluid must still be infused at a high rate during the pulse, which with respect to anesthetic drugs may have significant cardiovascular effects. Additionally, diastole phased infusion requires complex equipment to sense and phase the high-infusion-rate pulses in synch with diastole, which may vary during infusion. There is therefore a need for infusion methods and devices to overcome streaming which deliver fluid at lower infusion rates and/or independent of diastole.

Moreover, during traditional intra-aeterial infusions, the uptake of the drug by the tissue is determined by the biological properties of the drug. For example, in the brain there are specific transport mechanisms that buffer high concentrations of drugs injected into the carotid artery, such that tissue concentrations of the drug are not increased. The method of drug infusion described herein utilizes the physical and biological properties of the drug to define the characteristics of the pulse.

### SUMMARY OF THE INVENTION

This invention overcomes the shortcomings in the art by generally providing methods, devices, and software products for use in infusion therapy in a manner less prone to streaming, provides greater efficient use of the fluid, and with respect to drug infusion, in a manner providing greater efficient uptake to a targeted organ, such as the brain, and less systemic side affects.

In one aspect of the invention, a method of infusing a fluid into a blood vessel is provided that includes the step of administering the fluid into the blood vessel in programmed pulses. The programmed pulse characteristics may be defined by programmed pulse variables that include a flow rate for the drug-containing fluid, a frequency, and a duration. Values of the programmed pulse variables may be determined based at least in part on a fluid property of the fluid to be infused that is relevant to streaming, blood flow in the blood vessel to be infused, a catheter size, or a patient profile. In one embodiment, the fluid is administered in programmed pulses independent of diastole.

The methods described herein may be used in a variety of blood vessels. The methods are particularly useful in treating conditions associated with a patient's head, such stroke after thrombosis, cancer, cerebral vasospasm, infection, and localization of brain function. In these instances, the fluid may be delivered in programmed pulses into a patient's carotid artery. In one embodiment of the invention, the drug is administered at a fluid flow rate of about 2% to about 5% of arterial blood flow of the carotid artery. In one embodiment, the fluid is administered in programmed pulses at a duration of about one to five seconds.

Treatment generally relates to the discovery and application of remedies to manage or care for an injury or disease. The invention as described herein may be used to produce clinical imaging using X-rays, ultrasound, computed tomography, magnetic resonance, radionuclide scanning, thermography, etc., in connection with surgical procedures, such as to administer an anesthetic, or in connection with non-invasive treatments, such as to administer chemotherapy to tread cancer. It is reasonably understood that those skilled in the art may apply the present invention in a variety of treatments. The examples provided herein are therefore merely for illustration and not to be viewed as limitations.

In one embodiment, the fluid property of the fluid to be infused that is relevant to streaming includes a static fluid property or a kinetic fluid property. Alternatively, or in addition, where the fluid is a drug, the fluid property of the fluid to be infused that is relevant to streaming includes a pharmacological property of the drug. Additionally, where the fluid is a drug, the fluid may be administered in programmed pulses at the duration based on a transfer rate of the drug across a blood brain barrier or at the frequency based on a rate of elimination of the drug from a brain. In one embodiment, where the fluid is a drug, the concentration of the drug is determined based on a kinetic property of the drug and/or based on a therapeutic index of the drug.

In one aspect of this invention, a device for administering a fluid in programmed pulses, i.e., a programmed pulse infusion device, is provided. The programmed pulse infusion device includes a pump, a controller with associated memory interfacing with the pump, and a fluid reservoir containing a fluid feeding the pump. The controller provides a drive signal for the pump to deliver the fluid to be infused into a blood vessel in programmed pulses that are defined by programmed pulse variables, which include a fluid flow rate, a frequency, and a duration. The values of the programmed pulse variables may be determined based at least in part on a fluid property of the fluid to be infused relevant to streaming, blood flow in the blood vessel to be infused, a catheter size, or a patient profile. The programmed pulse infusion device may deliver the fluid in programmed pulses independent of diastole and for infusion into a carotid artery.

In one embodiment, the values of the programmed pulse variables base on the fluid property of the fluid to be infused relevant to streaming includes a static fluid property and/or a kinetic fluid property. Where the fluid is a drug, the fluid property of the fluid to be infused relevant to streaming may be a pharmacological property of the drug. Additionally, where the fluid is a drug, the duration may be based on a transfer rate of the drug across a blood brain barrier and the frequency may be based on a rate of elimination of the drug from a brain.

In one embodiment, the programmed pulse infusion device includes a control solenoid that interfaces with the controller. The controller may provide an actuating signal to the solenoid to deliver the fluid in programmed pulses. In this instance, the pump may be a pneumatic actuated pump. The programmed pulse infusion device may also include a bubble trap, which may be used for removing air bubbles from the fluid being delivered and may include a pressure sensor interfacing with the controller for observing the fluid delivery pressure. In one embodiment, the fluid reservoir is a syringe and the pneumatic actuated pump includes a plunger that is extended by an actuating force toward the syringe to deliver the fluid from the syringe in programmed pulses.

In one aspect of this invention, a software product or program code is provided on a computer readable medium that when executed enables a user to determine values of programmed pulse variables for infusing the fluid in a blood vessel in programmed pulses that are defined by the programmed pulse variables that include a fluid flow rate, a frequency, and a duration. The values of the programmed pulse variables may be determined at least in part on a fluid property of the fluid to be infused that is relevant to streaming, blood flow in the blood vessel to be infused, a catheter size, or a patient profile. The values of the programmed pulse variables may be determined independent of diastole. In one embodiment, the program code enables users to determine values of the programmed pulse variables for infusing the fluid into a carotid artery.

In one embodiment, the fluid property of the fluid to be infused that is relevant to streaming includes a static fluid property or a kinetic fluid property, and where the fluid is a drug, the fluid property of the fluid to be infused relevant to streaming may include a pharmacological property of the drug. Additionally, where the fluid is a drug, the drug may be infused in programmed pulses in a carotid artery to treat stroke after thrombosis, cancer, cerebral vasospasm, infection, or localization of brain function.

In one aspect of this invention, a computer system is provided that includes a controller and associated computer memory, and a computer readable medium that is accessible to the controller. Stored on the computer readable medium may be a database or databases including data of fluid properties for a fluid that may be infused into a blood vessel relevant to streaming, and values of programmed pulse variables based on a patient profile, a blood flow rate in the blood vessel, a fluid property for the fluid relevant to streaming, and/or a catheter size. The database may be accessed or is accessible to enable users to determine the values of programmed pulse variables for infusing the fluid in the blood vessel in programmed pulses that are defined by the programmed pulse variables that include a fluid flow rate, a frequency, and a duration.

In one embodiment, the values of programmed pulse variables may be based on a fluid property for the fluid relevant to streaming that may include a static fluid property or a kinetic fluid property. Where the fluid is a drug, the values of programmed pulse variables may be based on a fluid property for the fluid relevant to streaming that includes a pharmacological property of the drug. Additionally, where the fluid is a drug, the duration of a programmed pulse may be based on a transfer rate of the drug across a blood brain barrier and the frequency of a programmed pulse may be based on a rate of elimination of the drug from the brain.

In another aspect of the invention, methods, devices, and computer readable media are provided for infusing a fluid into a blood vessel by reducing blood flow in the blood vessel and administering the fluid into the blood vessel in programmed pulses defined by programmed pulse variables comprising a fluid flow rate, a frequency, and a duration. The blood flow may be reduced for less than about 20 seconds and may be reduced from about 0% to about 50% of a baseline blood flow.

Blood low may reduced with at least one occlusion catheter and fluid may be administered with an infusion catheter. Blood flow may also be reduced and fluid administered with a multiple lumen catheter that includes at least one first lumen for infusing fluid into the blood vessel and at least one second lumen for reducing blood flow in the blood vessel. The multiple lumen catheter may be a side-by-side catheter or one in which the multiple lumens of the multiple lumen catheter are disposed in a co-axial arrangement. Fluid may be administered at a fluid flow rate of about 2% to about 5% of a baseline arterial blood flow of a carotid artery and/or in programmed pulses at the duration of about one to five seconds.

### BRIEF DESCRIPTION OF THE FIGURES

The invention is illustrated in the figures of the accompanying drawings, which are meant to be exemplary, and not limiting, in which like references refer to like or corresponding parts, and in which:

FIG. 1 is a sectional view of a blood vessel showing the streaming effect on a fluid in the blood vessel;

FIG. 2 is a flowchart of a method for administering a fluid in programmed pulses, according to an embodiment of this invention;

FIGS. 3a and 3b are graphical representations of programmed pulses according to an embodiment of this invention;

FIG. 4 is a block diagram of a programmed pulse delivery device according to an embodiment of this invention; and

FIG. 5 is a block diagram of a programmed pulse delivery device with a pneumatic actuated pump according to one embodiment of this invention.

FIGS. 6a-c are bivariate-scattergrams showing the performance of Medfusion 2010i infusion pump against the resistance imposed P-50 arterial catheter. The pump was tested at 4 set volume infusion rates, 6, 12, 24, 48 ml/hr. The each flow rate was tested three times. There was a positive linear correlation between the set, the measured and the displayed volumes.

FIG. 7 is a bivariate-scattergram showing the effects of bolus dose on the total dose required to produce 5 minutes of EEG silence.

FIGS. 8a-c are videomicroscopy images taken 1 s before (3-a), during (3-b), and 1 s after (3-c) the injection of 0.1 ml bolus of propofol. A single artery lies transversely at the bottom of the picture and multiple veins drain the arterial irrigation. Under green illumination blood appears to be black in color, while propofol appears as white. Note that the bolus of 0.1 ml completely displaces the blood contained in the artery and most of the veins. This displacement is exceedingly transient. The bolus of the drug is washed out within 1 sec. of intracarotid injection.

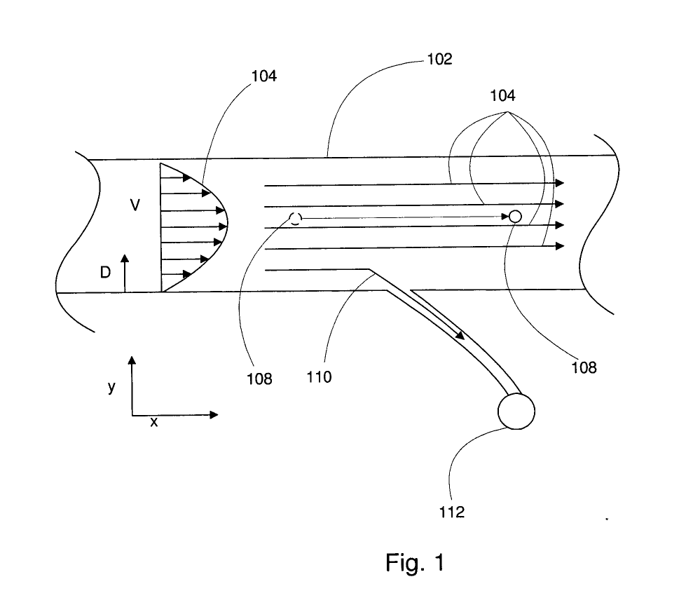

### DETAILED DESCRIPTION OF THE INVENTION

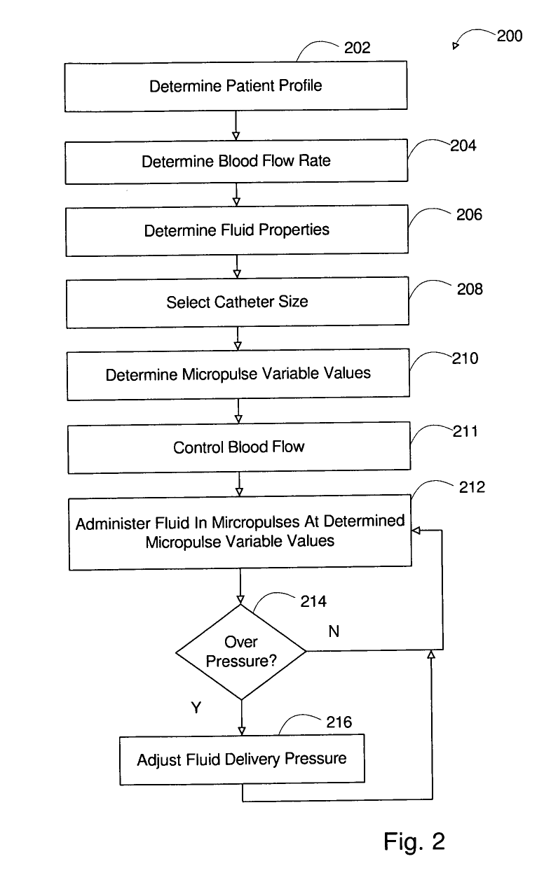

Referring to FIG. 2, a method for infusing or administering a fluid in programmed pulses, according to an embodiment of this invention, begins with determining a patient profile, step 202. A programmed pulse is generally a small volume of a fluid, such as a drug, in relatively short bursts, or otherwise at a low rate of infusion. A fluid denotes a substance in a form that takes the shape of its container, such as a substance in a liquid state. The patient profile generally refers to a characteristic or characteristics that may be useful in assessing a patient, such as weight, blood pressure, heart rate, temperature, etc. The profile is generally determined to assess the general condition of the patient, and to determine, in the instance the fluid is a drug, the dosage to be used for the infusion therapy. A drug generally denotes any substance that may be used in connection with a medical therapy, such as a pharmaceutical substance or composition, a medicine, a contrast agent used in X-Ray or magnetic resonance imaging (“MRI”), such as dyes, etc. A drug profile refers to the kinetic properties of drug delivery across the blood-brain border.

The patient profile may be determined with appropriate devices, such as a weight scale, blood pressure cuff, heart rate monitor, thermometer, etc., which are independent of the device for programmed pulse delivery of a fluid, e.g., the programmed pulse delivery device. In this instance, the patient profile or information related thereto, such as a dosage, may be noted for use in administering a fluid in programmed pulses as described herein, or input into the programmed pulse delivery device with an input device, such as a keypad or keyboard incorporated in or connected to the programmed pulse delivery device. Alternatively, the devices for determining the patients profile may interface with the programmed pulse delivery device, to provide patient profile data directly to the device.

In one embodiment, the blood flow rate of a blood vessel to be infused, e.g., the blood vessel that the fluid will be administered to, is determined, step 204. The blood flow rate may be determined in a variety of ways, such as with a blood flow sensor. The blood flow sensor may interface with the programmed pulse delivery device to provide the blood flow rate data directly to the programmed pulse delivery device. The blood flow sensor may be a stand-alone device independent of the programmed pulse delivery device. Blood flow data derived with the stand-alone sensor may similarly be noted for use in administering the fluid in programmed pulses or input into the programmed pulse delivery device with the input device. Alternatively, blood flow may be determined by estimating the blood flow based on the patient's profile. For example, blood flow data for a blood vessel, such as the carotid artery, for a plurality of patient profiles may be compiled and used to estimate or extrapolate the blood flow of the blood vessel for a particular patient based on the particular patient's profile. The compiled data for the plurality of patient profiles may be provided in a chart that may be used by medical professionals to manually estimate the blood flow or in database stored on a computer readable medium for estimating the blood flow with a programmed pulse delivery device.

Fluid properties relevant to streaming for the particular fluid to be infused may then be determined, step 206. Relevant fluid properties include static fluid properties, such as a fluid density, specific weight, specific gravity, viscosity, elasticity, etc., or kinetic properties. Where the fluid is a drug, the relevant fluid properties include pharmacological properties of the drug. Kinetic properties, generally relate to the transport or capability of being transported in the blood stream, and pharmacological properties relate to the effect or usefulness of the drug to be infused. For example, relevant kinetic properties may include streaming or anti-streaming properties, the typical volume rate of infusion, etc. Pharmacological properties may include, with respect to a particular fluid, data relating to the concentration of the drug in the blood vessel and the concentration of the blood during re-circulation, protein binding, cerebral transit time, bio-phase equilibrium time, blood:brain partition coefficient, transfer rate across the blood:brain barrier, blood:brain transfer profile of the drug, active transport, drug formulation, rate of elimination or efflux from the brain, therapeutic index or concentration, maximum tolerable concentration, receptor efficacy, local metabolism in the brain, etc. The relevant fluid properties for fluids may be provided in chart form or in databases stored on a computer readable medium, which may be input or accessible to the programmed pulsed delivery device.

A catheter size for programmed pulsed infusion may then be selected, step 208. The catheter size is generally selected in accordance with the size of the blood vessel that is to be infused. The catheter size may also be input or accessible to the programmed pulsed delivery device.

At step 210, the value of a programmed pulse variable or variables are determined. Programmed pulse variables are variables that generally define a programmed pulse. Programmed pulse variables may include variables related to the timing of the programmed pulse, the volume of fluid infused, the pressure at which the fluid will be infused, etc. In one embodiment, programmed pulse variables that define a programmed pulse include a fluid flow rate, a frequency, and duration. A programmed pulse variable, in the instance the fluid is a drug, may include a drug concentration where for example the drug is included in a solution or suspension. Additionally, programmed pulse variables may include fluid delivery pressure.

The value or values of programmed pulse variables that define programmed pulses may be determined in a variety of ways. In one embodiment, the values are determined based at least partially on a fluid property relevant to streaming, such as a static fluid property, kinetic property, or where the fluid is a drug, based on a pharmacological property. In another embodiment, the values of programmed pulse variables are be based at least partially on the blood flow rate in the blood vessel to be infused. In yet another embodiment, the values are based at least partially on the patient profile. The values of programmed pulse variables are preferably determined by or in connection with the programmed pulse delivery device based on the patient profile, a fluid property relevant to streaming, or a blood flow rate in the blood vessel to be infused, or a combination thereof. For example, the programmed pulse delivery device may access fluid property data for the particular fluid to be administered and may determine the values of programmed pulse variables that will be used in defining programmed pulses for infusing the particular fluid. In one embodiment, a database including a set or sets of predefined values of programmed pulse variables, for a variety of patient profiles, blood flow rates, fluid properties, or catheter sizes, are stored on a computer readable medium. The programmed pulse delivery device may access the database to determine the appropriate values by looking up and/or computing the values based on the patient profile, blood flow rates, fluid properties relevant to streaming, or catheter size for a particular situation.

The fluid may then be administered in programmed pulses accordingly, step 212. The fluid may be administered in programmed pulses having programmed pulse variables with values determined or computed based on the patient profile, blood flow rate of the blood vessel to be infused, a fluid property relevant to streaming for the fluid to be infused, the catheter size to be used for infusing the fluid, or a combination thereof. In one embodiment, the fluid is administered into a blood vessel in programmed pulses having a flow rate of about 2% to 5% of the blood flow in the blood vessel to be infused and/or for a duration of 1-5 seconds. In one embodiment, the fluid is solution or suspension including a drug with a concentration that is determined based on the dosage and/or the drugs kinetic properties. Alternatively, the drug concentration may be determined based on a therapeutic index or concentration for the drug, or a blood:brain transfer profile. In one embodiment, the fluid is a drug and the duration is based on the transfer rate across the blood:brain barrier or blood:brain transfer profile or coefficient of the drug, receptor efficacy, or local metabolism in the brain. In another embodiment, the fluid is a drug and the frequency of the programmed pulses is determined based on the rate of elimination of efflux of the drug from the brain and/or the concentration of the drug if in a solution or suspension.

In one embodiment, the fluid delivery pressure is observed to prevent an overpressure condition, step 214. An overpressure condition generally refers to a condition in which the fluid delivery pressure exceeds a working pressure or pressure limit. A working pressure is generally a pressure less than the pressure limit, such as the pressure limit with an appropriate safety factor applied. For example, a working pressure for a pressure limit of 800 mm Hg with a Safety factor of 1.5 is 800 mm Hg./1.5=533 mm Hg. If at step 214 an overpressure condition is observed, the fluid delivery pressure may be adjusted, step 216, or the infusion may be stopped.

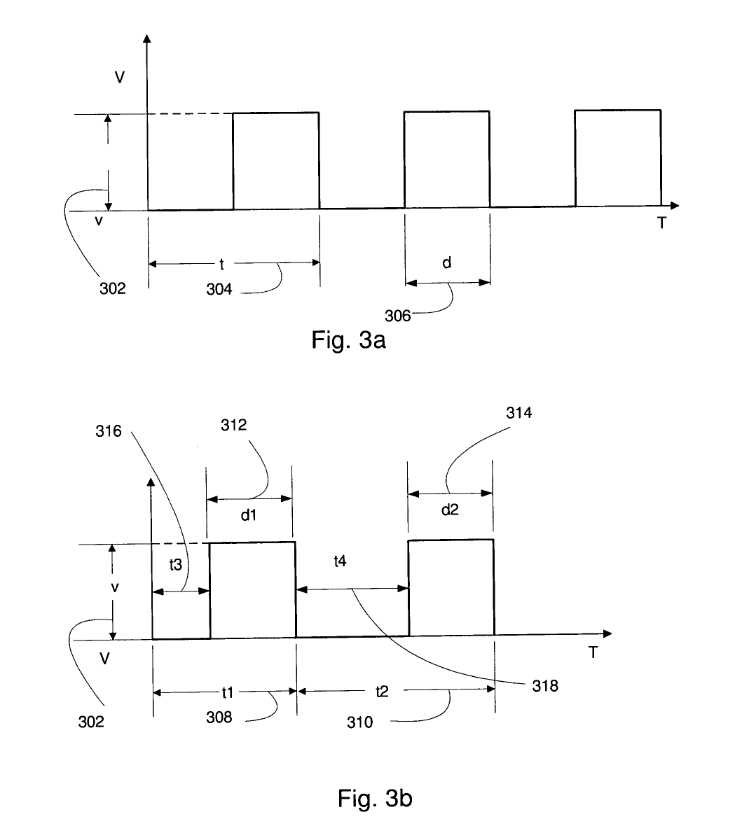

Referring to FIG. 3a, a graphical representation of a series of programmed pulses, according to one embodiment of this invention, is shown in terms of fluid flow (V) and time (T). A programmed pulse can generally be described as having a fluid flow rate (v) 302, a frequency (f), and duration (d) 306. The frequency may be described in terms of programmed pulses/time (t), e.g., 1.5 programmed pulses per second, etc. The amount of fluid administered per pulse is the flow rate (v) multiplied by the duration (d). The total amount of fluid administered is the amount of fluid administered per pulse multiplied by the frequency (f) and the total infusion time. In one embodiment, programmed pulses are administered at a fluid flow rate of about 5% of the blood flow in the blood vessel being infused and for a duration (d) of about 1 second until the desired dosage is achieved. Referring to FIG. 3b, the programmed pulses may be administered in a variable pattern. For example, the frequency (f) may vary for times (t1) 308 and (t2) 310. The duration as shown in with (d1) 312 and (d2) 314, the time frame between the pulses as shown with (t3) 316 and (t4) 318, and the flow rate (v) may vary over the infusion period. The fluid flow rate (v), frequency (f), and duration (d) programmed pulse variables may be determined or computed in either the fixed or variable programmed pulse embodiments based on the patient profile, blood flow rate, a fluid property relevant to streaming, catheter size, or a combination thereof.

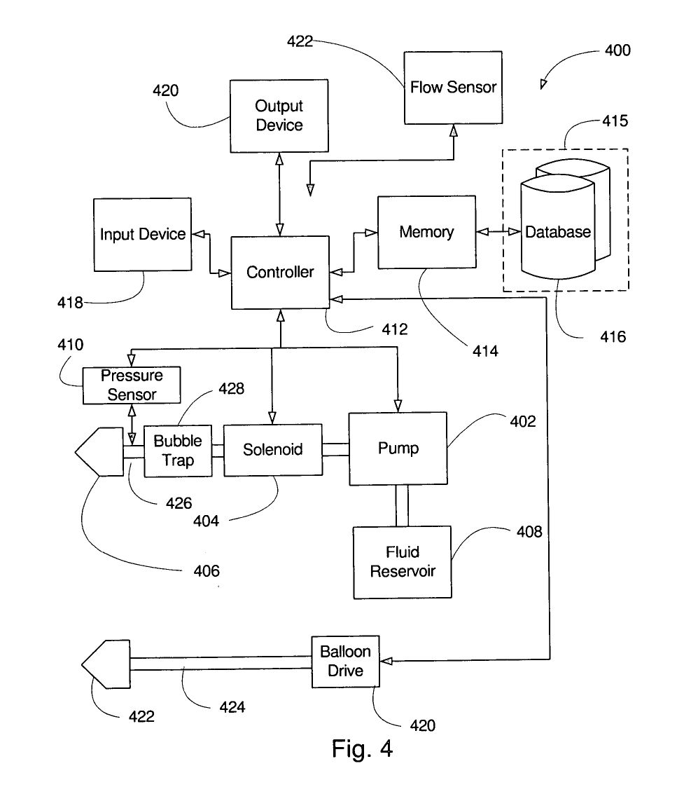

Referring to FIG. 4, a programmed pulse delivery device 400, in one embodiment, includes a pump 402, a controller 412 with associated computer memory 414, and a fluid reservoir 408. The controller generally provides a drive signal to drive the pump 402 for delivering the fluid in programmed pulses through an orifice, such as a catheter 406. The controller may be a micro-controller or processor that is capable of providing the drive signal to provide programmed pulse fluid infusion as described herein. The controller may be programmed or program code may be provided enabling the controller to access relevant data input by a user, such as the patient profile, blood flow rate, the particular fluid to be infused, fluid properties relevant to streaming, catheter size, values for programmed pulse variables, etc., for use in providing the drive signal. Alternatively, or in addition, the controller may access relevant data stored on at least one database, such as data of blood flow rates based on patient profiles, fluid properties relevant to streaming for particular fluids and concentrations, values for programmed pulse variables based on blood flow in blood vessel to be infused, fluid properties relevant to streaming, patient profiles, catheter size, or a combination therefore.

In one embodiment, the computer memory 414 has associated therewith at least one database 416. A database or databases may include data of blood flow for a blood vessel or vessels for a plurality of patient profiles, fluid property data relevant to streaming for particular fluids that may be infused, such as data of kinetic and pharmacological properties of a drug, values of programmed pulse variables based on a patient profile, blood flow rate, a fluid property relevant to streaming, and/or a catheter size. The databases may be stored on a computer readable medium that is accessed by the controller to provide the drive signal for programmed pulsed fluid infusion.

In one embodiment, the controller, computer memory, and a computer readable medium 415 are provided in a standalone computer, such as a personal computer, a special purpose computer, etc., that interfaces with the pump 402 to provide the drive signal for programmed pulsed infusion. The database or databases may be stored locally at the stand-alone computer, or remotely, such as on a server computer connected to the standalone compute over a communication network, such as a local area network (“LAN”), wide area network (“WAN”), etc. The databases may be stored on computer readable medium 415, such as a hard drive, optical media, magnetic tape, etc. Additionally, the computer readable medium 415 may include program code that when executed determines the values of programmed pulse variables based on a patient profile, blood flow in the blood vessel to be infused, fluid properties relevant to streaming for particular fluids, catheter size, or a combination thereof. In one embodiment, the stand-alone computer does not interface with the pump. In this instance, the values of programmed pulse variables may be determined and displayed to the user, which may then be input into a stand-alone pump capable of delivering a fluid in programmed pulsed fashion in accordance with the values of programmed pulse variables determined with the stand-alone computer.

In one embodiment, information, such as the patient profile, the particular fluid being administered, and the catheter size may be provided to the controller with an input device 418, such as a keypad, keyboard, mouse, touch pad, etc. The controller may display information with an output device 420. The output device may be a liquid crystal display (“LCD)”, a cathode ray tube (“CRT”) monitor, etc., which may also be a touch screen data input device. In one embodiment, the controller interfaces with a flow sensor 422, which provides the blood flow rate in the blood vessel to be infused. A pressure sensor 410 interfacing with the controller may also be included for observing fluid delivery pressure.

The pump 402 may be any type of pumping apparatus, including, but not limited to a pneumatic actuated pump, etc. In one embodiment, the programmed pulse delivery device includes a control solenoid 404 that is actuated with a signal from the controller to deliver the fluid in programmed pulses as described herein. The device may also include a bubble trap 428 for removing air bubbles from the fluid being infused.

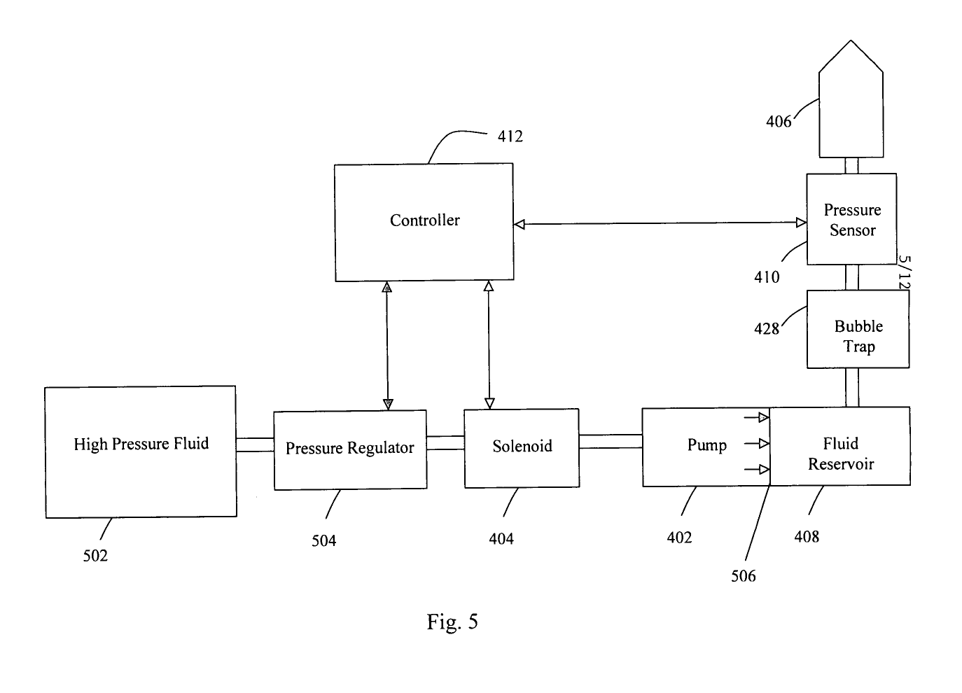

Referring to FIG. 5, a programmed pulse delivery device with a pneumatic actuated pump according to one embodiment of this invention includes a high-pressure actuating fluid source 502, which provides the actuating force for the pneumatic pump 402. The high-pressure actuating fluid source 502 may be any compressed gas, such as air, nitrogen, oxygen, etc. In one embodiment, the programmed pulse delivery device includes a pressure regulator 504 for regulating the high-pressure actuating fluid to a desired pressure, such as a working pressure or a pressure limit. The pressure regulator 504 may be set and adjusted manually or automatically by the controller 412. The control solenoid 404 receives the drive signal from the controller 412 to release the high-pressure or regulated actuating fluid to the pneumatic pump 402 for the pneumatic pump 402 to deliver the fluid in programmed pulses as described herein. In one embodiment, the pneumatic pump 402 includes a plunger 506 that extends with the application of the actuating force created by the high or regulated pressure actuating fluid. The plunger 506 extends an amount corresponding to the variables of the programmed pulses into or toward the fluid reservoir to expel the fluid from the fluid reservoir 408, such as a syringe. The expelled fluid delivered from the fluid in the reservoir in programmed pulses. In one embodiment, the syringe includes a Luer lock.

Referring back to FIG. 2, In one embodiment, fluid is administered or infused in programmed pulses with or without also controlling blood flow in the target blood vessel, at step 211. Controlling generally includes arresting, reducing, increasing, or a combination thereof. Blood flow, for example, may be reduced to 0%-50% of baseline, or preferably 25-35% of baseline, for the duration of one or more pulses or a portion thereof. When fluid is administered with a plurality of pulses, blood flow may be reduced during one or more of the pulses. Blood flow may be controlled in a variety of ways, including pharmacological means, mechanical means, or a combination thereof. For example, blood flow may be reduced with adenosine and/or esmolol, or a balloon or other occlusion catheter inserted into a blood vessel to control blood flow during pulse infusion.

In one embodiment, blood flow is controlled with a plurality of catheters: at least one for infusing otherwise administering the fluid into the target vessel and at least one for occluding the blood vessel, e.g., to reduce or arrest blood flow therein. The plurality of catheters may be used in different sections of the target blood vessel or in the same section of the blood vessel. For example, a catheter may be introduced into the carotid artery for infusing fluids targeted for the brain and one or more balloon catheters may be introduced downstream of the blood flow, such as in the posterior communicating artery (PCA), the middle cerebral artery (MCA), the anterior cerebral artery (ACA), etc., to reduce or arrest blood flow in the carotid artery. Infusion and balloon catheters may be introduced into the carotid artery and the balloon catheter inflated either, e.g., about 1 mm to about 10 cm downstream or upstream of the infusion catheter to similarly reduce or arrest blood flow therein.

Referring to FIG. 4, in one embodiment, blood flow is reduced or arrested with a steerable device, e.g., a double- or muliple-lumen balloon catheter having at least one lumen, e.g., a first lumen, for infusing fluids into a blood vessel 426 and at least one lumen, e.g., a second lumen 424, for inflating and deflating the balloon 422. The multiple lumens may be fixed or movable in relation to each other and may be disposed in a side-by-side or a co-axial arrangement. The balloon 422 may be either distal or proximal to the distal end of the infusion catheter. The first lumen 426 of the double-lumen assembly may be a micro-catheter and the second lumen 424 of the double-lumen assembly is a larger lumen for inflating and deflating the balloon 422, and wherein the balloon can be rapidly inflated and deflated. In one embodiment, the proximal double-lumen assembly is about 4-5.5 French in diameter for the first 80-100 cm. In the distal end, preferably, the distal 8-15 cm, the diameter of the proximal double-lumen assembly is gradually narrowed to about 2-3 French. The micro-catheter is extended beyond the distal end of the balloon for a variable length, preferably, 1-10 cm beyond the distal end of the balloon. In one embodiment, the micro-catheter is about 1-2 French in diameter, preferably, about 1.2-1.5 French in diameter. In another embodiment, the balloon is about 1-1.5 cm in length. The balloon of the present invention can be rapidly inflated or deflated, optimally, in about 1 second.

The steerable device, e.g., the catheter, is preferably made of materials which render the steerable device strong enough to withstand repeated inflation and deflation of the balloon, flexible enough to negotiate the curve of blood vessels and having low frictional resistance and thrombogenic potential. The material may be any suitable material with high tensile strength, such as, Teflon, nylon, polyurethane, and polyethylene. In one embodiment, to increase maneuverability and decrease the risk of thromboembolism, the steerable device has a surface coating. In a preferred embodiment, the surface coating is a hydrophilic surface coating.

In one aspect, the present invention provides a drug delivery system which comprises a steerable device and a balloon drive 420, wherein the steerable device comprises a proximal double-lumen assembly and a balloon 422, wherein the first lumen 426 of the double-lumen assembly is a micro-catheter and the second lumen 424 of the double-lumen assembly is a larger lumen for inflating and deflating the balloon 422, and wherein the balloon can be rapidly inflated and deflated. In one embodiment, the proximal double-lumen assembly is about 4-5 French in diameter for the first 80 cm. In the distal end, preferably, the distal 10 cm, the diameter of the proximal double-lumen assembly is gradually narrowed to about 2-3 French. The micro-catheter is extended beyond the distal end of the balloon for a variable length, preferably, 1-10 cm beyond the distal end of the balloon. In a preferred embodiment, the micro-catheter is about 1-2 French in diameter, more preferably, about 1.2-1.5 French in diameter. In another embodiment, the balloon is about 1-1.5 cm in length. The balloon of the present invention can be rapidly inflated or deflated, optimally, in about 1 second.

The steerable device, e.g., a catheter, is preferably made of materials which render the steerable device strong enough to withstand repeated inflation and deflation of the balloon, flexible enough to negotiate the curve of blood vessels, and having low frictional resistance and thrombogenic potential. The material may be any suitable material with high tensile strength, such as, Teflon, nylon, polyurethane and polyethylene. In one embodiment, to increase maneuverability and decrease the risk of thromboembolism, the steerable device has a surface coating, preferably, a hydrophilic coating.

The inflation and deflation of the balloon is controlled by the balloon drive 420. The balloon drive 420 may be any device which is capable of rapidly inflating or deflating the balloon, which may include a fluid pump and a controller, or for similarly operating an occlusion catheter. In one embodiment, the balloon drive inflates or deflates the balloon in less than 20 seconds, preferably, in less than 5 seconds, and more preferably, in about 1 second. The balloon drive may use any suitable liquid or gas to inflate the balloon. In a preferred embodiment, the balloon is inflated by a radio-opaque low-viscosity fluid. The fluid based balloon distention mechanism decreases the time required to inflate a balloon.

The drug delivery system may further comprise a computerized device, e.g., a controller to control the balloon drive 420. The balloon drive 420 may share a common controller 412 or use independent controllers. In either event, the controller(s) cooperate to inflate the balloon and pulse the fluid as discussed above in concert with each other. The computerized device may be any computing system suitable for controlling the balloon drive. The computerize device may be a stand-alone computer, which is functionally connected to the balloon drive, or integrated with the balloon drive. In either case, the computer is capable of receiving external and/or internal input and transferring the input into signals to control the behavior of the balloon drive. The input information may be any information that may contribute to the manipulation of the function of the balloon drive. The primary inputs are parameters used by the computerized device to control the balloon drive, such as the frequency, duration and volume of inflation/deflation.

The present invention further provides a method for the localized delivery of an agent to a target location within a subject, comprising the steps of: (1) partially or completely arresting blood flow to the target location for a short period of time; (2) delivering the agent in bolus to the target location; and (3) partially or completely restoring blood flow to the target tissue, wherein the blood flow is arrested by occluding the artery to the target tissue.

As used herein, the “subject” is an animal, preferably a mammal including, without limitation, a cow, dog, human, monkey, mouse, pig or rat. The term “agent,” as used herein, shall include any protein, polypeptide, peptide, nucleic acid (including DNA, RNA, and genes), antibody and fragment thereof, molecule, compound, antibiotic, drug and any combinations thereof. The agent of the present invention may have any activity, function or purpose. By way of example, the agent may be a diagnostic agent, a labeling agent, a preventive agent, or a therapeutic or pharmacologic agent.

As used herein, a “diagnostic agent” is an agent that is used to detect a disease, disorder or illness or is used to determine the cause thereof. As further used herein, a “labeling agent” is an agent that is linked to, or incorporated into, a cell or molecule, to facilitate or enable the detection or observation of that cell or molecule. By way of example, the labeling agent of the present invention may be an imaging agent or detectable marker and may include any of those radioactive labels known in the art. For instance, the labeling agent may be a radioactive marker, including a radioisotope, such as a low-radiation isotope. The radioisotope may be any isotope that emits detectable radiation, and may include ³⁵S, ³²P, ³H, radioiodide (¹²⁵I- or ¹³¹I-) or 9 mTc-pertechnetate (⁹⁹ mTcO₄⁻). Radioactivity emitted by a radioisotope can be detected by techniques well known in the art.

Additionally, as used herein, the term “preventive agent” refers to an agent, such as a prophylactic, that helps to prevent a disease, disorder or illness in a subject. As further used herein, the term “therapeutic” refers to an agent that is useful in treating a disease, disorder or illness (e.g., a neoplasm) in a subject. In one embodiment, the anti-neoplasm agent used in a method to prevent and treat a neoplasm is an antibody. In a preferred embodiment, the antibody is preferably a mammalian antibody (e.g., a human antibody) or a chimeric antibody (e.g., a humanized antibody). More preferably, the antibody is a human or humanized antibody. As used herein, the term “humanized antibody” refers to a genetically-engineered antibody in which the minimum portion of an animal antibody (e.g., an antibody of a mouse, rat, pig, goat or chicken) that is generally essential for its specific functions is “fused” onto a human antibody. In general, a humanized antibody is 1-25%, preferably 5-10%, animal; the remainder is human. Humanized antibodies usually initiate minimal or no response in the human immune system. Methods for expressing fully human or humanized antibodies in organisms other than human are well known in the art (see, e.g., U.S. Pat. No. 6,150,584, Human antibodies derived from immunized xenomice; U.S. Pat. No. 6,162,963, Generation of xenogenetic antibodies; and U.S. Pat. No. 6,479,284, Humanized antibody and uses thereof). In one embodiment of the present invention, the antibody is a single-chain antibody. In a preferred embodiment, the single-chain antibody is a human or humanized single-chain antibody. In another preferred embodiment of the present invention, the antibody is a murine antibody.

In one embodiment of the present invention, the therapeutic agent, such as an anti-neoplasm agent, may be a nucleic acid (e.g., plasmid). The nucleic acid may encode or comprise at least one gene-silencing cassette, wherein the cassette is capable of silencing the expression of genes that are essential or important for the survival or proliferation of pathogens or neoplastic cell. It is well understood in the art that a gene may be silenced at a number of stages including, without limitation, pre-transcription silencing, transcription silencing, post-transcription silencing, translation silencing and post-translation silencing. The nucleic acid may also encode polypeptides or other types of biological molecules which are capable of compensating or correcting a defect in a subject.

In one embodiment of the present invention, the gene-silencing cassette encodes or comprises a post-transcription gene-silencing composition, such as antisense RNA or RNAi. Both antisense RNA and RNAi may be produced in vitro, in vivo, ex vivo, or in situ.

For example, the therapeutic agent of the present invention, e.g., an anti-neoplasm or anti-infection agent, may be an antisense RNA. Antisense RNA is an RNA molecule with a sequence complementary to a specific RNA transcript, or mRNA, whose binding prevents further processing of the transcript or translation of the mRNA. Antisense molecules may be generated synthetically or recombinantly with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein, et al., Oligodeoxynucleotides as inhibitors of gene expression: a review. Cancer Res., 48:2659-68, 1998).

Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes (e.g., U.S. Pat. No. 6,448,080, Antisense modulation of WRN expression; U.S. Patent Application No. 2003/0018993, Methods of gene silencing using inverted repeat sequences; U.S. Patent Application No., 2003/0017549, Methods and compositions for expressing polynucleotides specifically in smooth muscle cells in vivo; Tavian, et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J. Biol. Chem., e-publication ahead of print, 2003; Ghosh, et al., Role of superoxide dismutase in survival of Leishmania within the macrophage. Biochem. J, 369:447-52, 2003; and Zhang, et al., An anti-sense construct of full-length ATM cDNA imposes a radiosensitive phenotype on normal cells. Oncogene, 17:811-8, 1998).

In one embodiment, oligonucleotides antisense to a biological molecule, such as a member of the infection/neoplasm-related signal-transduction pathways/systems, may be designed based on the nucleotide sequence of the member of interest. For example, a partial sequence of the nucleotide sequence of interest (generally, 15-20 base pairs), or a variation sequence thereof, may be selected for the design of an antisense oligonucleotide. This portion of the nucleotide sequence may be within the 5′ domain. A nucleotide sequence complementary to the selected partial sequence of the gene of interest, or the selected variation sequence, then may be chemically synthesized using one of a variety of techniques known to those skilled in the art including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

Once the desired antisense oligonucleotide has been prepared, its ability to prevent or treat diseases, such as neoplasm, then may be assayed. For example, the antisense oligonucleotide may be administered to a subject, such as a mouse or a human, and its effects on the disease may be determined using standard clinical and/or molecular biology techniques, such as Western-blot analysis and immunostaining.

It is within the confines of the present invention that antisense oligonucleotides may be linked to another agent, such as an anti-infection, an anti-neoplastic drug, or an agent which facilitate the transportation of the antisense oligonucleotides into a cell (e.g., penetratin, transportan, pIsl, TAT, pVEC, MTS, and MAP). Moreover, antisense oligonucleotides may be prepared using modified bases (e.g., a phosphorothioate), as discussed above, to make the oligonucleotides more stable and better able to withstand degradation.

The therapeutic agent of the present invention also may be an interfering RNA, or RNAi, including small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi (see, e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAi is produced in vitro, synthetically or recombinantly. Methods of making and transferring RNAi are well known in the art (see, e.g., Ashrafi, et al., Genome-wide RNAi analysis of Caenorhabditis elegans fat regulatory genes. Nature, 421:268-72, 2003; Cottrell, et al., Silence of the strands: RNA interference in eukaryotic pathogens. Trends Microbiol., 11:37-43, 2003; Nikolaev, et al., Parc. A Cytoplasmic Anchor for p53. Cell, 112:29-40, 2003; Wilda, et al., Killing of leukemic cells with a BCR/ABL fusion gene RNA interference (RNAi). Oncogene, 21:5716-24, 2002; Escobar, et al., RNAi-mediated oncogene silencing confers resistance to crown gall tumorigenesis. Proc. Natl. Acad. Sci. USA, 98:13437-42, 2001; and Billy, et al., Specific interference with gene expression induced by long, double-stranded RNA in mouse embryonal teratocarcinoma cell lines. Proc. Natl. Acad. Sci. USA, 98:14428-33, 2001).

Once the desired RNAi has been prepared, its ability to prevent or treat diseases, such as neoplasm, then may be assayed. For example, the RNAi may be administered to a subject, such as a mouse or a human, and its effects on the disease may be determined using standard clinical and/or molecular biology techniques, such as Western-blot analysis and immunostaining.

It is within the confines of the present invention that an RNAi may be linked to another agent, such as an anti-infection, an anti-neoplastic drug, or an agent which facilitate the transportation of the antisense oligonucleotides into a cell (e.g., penetratin, transportan, pIsl, TAT, pVEC, MTS, and MAP). Moreover, an RNAi may be prepared using modified bases (e.g., a phosphorothioate), as discussed above, to make it more stable and better able to withstand degradation.

The agent may also be a pharmaceutical composition comprising the a therapeutic agent and a pharmaceutically acceptable carrier. The pharmaceutically acceptable carrier must be “acceptable” in the sense of being compatible with the other ingredients of the composition, and not deleterious to the recipient thereof. The pharmaceutically acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, viscosity-increasing agents, etc. If necessary, pharmaceutical additives, such as antioxidants, may also be added. Examples of acceptable pharmaceutical carriers include glycerin, lactose, magnesium stearate, saline, sodium alginate, sucrose, and water, among others.

The composition of the present invention may be prepared by methods well known in the pharmaceutical arts. For example, the composition may be brought into association with a carrier or diluent, as a suspension or solution. Optionally, one or more accessory ingredients (e.g., buffers, surface active agents, and the like) also may be added.

The pharmaceutical composition is provided in an amount effective to treat the disorder in a subject to whom the composition is administered. As used herein, the phrase “effective to treat the disorder” means effective to ameliorate or minimize the clinical impairment or symptoms resulting from the infectious disease or neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; or by limiting, suspending, terminating, or otherwise controlling the proliferation of cells in the neoplasm.

The amount of pharmaceutical composition that is effective to treat infectious diseases and neoplasia in a subject will vary depending on the particular factors of each case, including, for example, the type or stage of the infection or neoplasia, and the severity of the subject's condition. These amounts can be readily determined by the clinician.

In accordance with the method of the present invention, the pharmaceutical composition may be administered to a subject, either alone or in combination with one or more other therapeutic agents, such as antibiotics or antineoplastic drugs. Examples of antibiotics with which the pharmaceutical composition may be combined include, without limitation, penicillin, tetracycline, bacitracin, erythromycin, cephalosporin, streptomycin, vancomycin, D-cycloserine, fosfomycin, cefazolin, cephaloglycin, cephalexin, amphotericin B, gentamicin, tobramycin, kanamycin, and variants and derivatives thereof. Examples of antineoplastic drugs with which the pharmaceutical composition may be combined include, without limitation, carboplatin, cyclophosphamide, doxorubicin, etoposide and vincristine. The pharmaceutical composition of the present invention may also be administered to a subject together with an agent which is capable of improving the uptake of the pharmaceutical composition by the target tissue. For example, serotonin may be used to enhance arterial permeability and thus facilitate the transition of the therapeutic composition from artery to the target tissue.

Under certain circumstances, it is necessary to repeat the steps of (1)-(3) of the method of the present invention at least once. For example, the target tissue may be very sensitive to ischemic injury and thus shall not be subject to long-term blood occlusion. It is therefore preferable to repeat steps (1)-(3) such that, on the one hand, enough agents such as therapeutic drugs can be delivered to the target tissue; on the other hand, damages caused by ischemia-reperfusion may be minimized.

In one embodiment, the blood flow is arrested by inflating a balloon and restored by deflating the balloon. In another embodiment, the blood flow is arrested through a balloon together with a blood flow arresting pharmaceutical composition, such as adenosine and esmolol. Complete blood flow arrest is not always necessary for efficient drug delivery. A transient (e.g., 20-30 seconds) flow decrease to about 25% of baseline value is sufficient to enhance significantly the delivery of drug. In a preferable embodiment, the balloon is rapidly inflated and/or deflated, such as within about 1 second. In another embodiment, the duration of the balloon inflation is about 10-150 seconds. The inflation time depends on the ability of the tissue to with stand reduced blood flows. For organs like the brain the inflation time will be short (10-150 seconds) but for liver and heart it could be much longer (several minutes). It is desirable to employ a computer-controlled balloon drive to regulate the inflation and deflation of the balloon.

The agent may be delivered using a catheter. In one embodiment, the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated.

The agent delivered may be any therapeutic or diagnostic agent for the treatment or diagnosis of pathological conditions including, without limitation, agents for treating brain-related disorders, chemotherapeutic agents, and gene-therapy agents.

In one embodiment, the target location is in or close to a tissue in the subject, wherein the tissue has a pathological condition. Preferably, the target location is the artery in or near brain, a tumor, or a tissue in need of gene-therapy, such as carotid. In a preferred embodiment, the subject is a mammal, including human.

The devices and methods of the present invention are particular suitable for delivering drugs to the brain. The arteries in the brain are end-arteries, i.e., they do not join each other after they branch off from the parent arteries. Thus proximal arterial occlusion can effectively decrease blood flow in the distal regions of the brain. Furthermore, the devices and methods of the present invention may significantly improve cancer chemotherapy. Chemotherapeutic agents are generally poorly absorbed when given intra-arterially. The controlled-arterial occlusion drug delivery technique provided by the present invention will be very useful for efficient delivery of chemotherapeutic agents and thus decreasing the dose of chemotherapeutic agents needed and the systemic complications caused by these agents, which are generally highly toxic. Additionally, intra-arterial occlusion drug therapy could play a critical role in delivering gene therapy agents, such as viral vectors, liposomes and gene fragments.

The present invention also provides a method for the localized delivery of an agent to a target location within a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated; (2) incorporating the agent into the drug delivery system; and (3) delivering the agent to the target location.

The inflation and deflation of the balloon is controlled by the balloon drive, which, preferably, is controlled by a computerized device. The computerized device may be any computing system suitable for controlling a balloon drive. In one embodiment, the computerized device is a stand-alone computer system, with an input device, user-machine interface, and is functionally connected to the balloon drive. In another embodiment, the computerized device is a sub-component of a component of the drug delivery system, wherein the component further comprises the balloon drive. Depending on specific situations, such as the condition of the subject, the type of the target tissue, the purpose of the operation, the characteristic of the agent, different parameters should be used to control the behavior of the balloon drive and consequently, the inflation and deflation of the balloon. In one embodiment, the primary parameters used by the computerized device to control the balloon drive are the frequency, duration, and volume of inflation/deflation. The parameters may be manually inputted through a user-computer interface and an inputting device, or imported from a database, such as a medical expert system. The frequency of balloon inflation and deflation will be a function of a number of factors including, without limitation, the rate of efflux of the drug from the tissue, the duration of inflation, the type of the tissue, the type of the agent and the characteristics of reactive hyperemia in the tissue. The duration of the inflation will be a function of the risk of ischemic injury to the tissue, typically, between about 2-600 seconds, preferably, between about 5-100 seconds, more preferably, between about 15-60 seconds.

The balloon may be inflated by any suitable gas or liquid. In one embodiment, the balloon is inflated by fluid. The use of fluid will decrease the time required to inflate the balloon. Preferably, a radio-opaque low viscosity fluid is used to inflate the balloon because it will facilitate the imaging and monitoring of the performance of the balloon and the catheter.

In another embodiment, the agent used in the present method is a therapeutic or diagnostic agent, such as an agent for treating brain-related disorders, a chemotherapeutic agent, and a gene-therapy agent. To facilitate the effective delivery of the agent, it is desirable to have the balloon inflated to arrest partially or completely the blood flow to the target location. The target location is in or close to a tissue in the subject, wherein the tissue has a pathological condition, for example, the target location may be the artery (e.g., carotid) in or near brain, a tumor or a tissue in need of gene-therapy.

In a preferred embodiment, the agent is delivered to the target location in bolus or in pulses as discussed above. Computer simulations indicate that the efficacy of intra-arterial drug delivery is inversely affected by regional blood flow. For example, high blood flow creates a stable fluid flow system. A stable fluidic flow pattern can trap drugs within a sub-stream, resulting in streaming of drugs. Streaming generates heterogeneous distributions of drugs within the target tissue. There are variations in tissue drug concentrations as well as distribution after continuous infusions. Such unpredictability is therapeutically undesirable. Therefore, bolus delivery of drugs is more likely to generate predictable drug concentrations in the target tissue than infusions.

The present invention further provides a method for the localized delivery of an agent to a target location within a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated; (2) incorporating the agent into the drug delivery system; (3) occluding blood flow to the target location by inflating the balloon; (4) delivering the agent in bolus or in one or more pulses to the target location; and (5) deflating the balloon after a short period of time.

In one aspect, the present invention provides a method for the treatment of a pathological disorder in a subject, comprising the steps of: (1) partially or completely arresting blood flow to a target tissue for a short period of time; (2) delivering a therapeutic agent in bolus or in one or more pulses; and (3) partially or completely restoring blood flow to the target tissue, wherein the target tissue has a pathological condition and the blood flow is arrested by occluding the artery to the target tissue, and wherein the therapeutic agent is delivered into the target tissue or a location within the artery which is close to the target tissue. In one embodiment, the steps of (1)-(3) are repeated at least once to ensure sufficient drug delivery and minimize the ischemic injury. In another embodiment, the target tissue is brain, a tumor, or a tissue in need of gene-therapy.

The blood flow may be arrested by inflating a balloon and restored by deflating the balloon. In one embodiment, the balloon is inflated or deflated in about 1 second using a balloon drive, which is subsequently controlled by a computerized device.

The therapeutic agent may be delivered using a catheter. In one embodiment, the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

In another aspect, the present invention further provides a method for the treatment of a pathological disorder in a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating a therapeutic agent into the drug delivery system; and (3) delivering the therapeutic agent to a target location, wherein the target location is in or close to a target tissue in the subject, wherein the target tissue has a pathological condition. In one embodiment, the catheter is made of a material with high tensile strength, such as Teflon, nylon, polyurethane, and polyethylene. The catheter may have a surface coating, preferably a hydrophilic surface coating. In another embodiment, the balloon inflation partially or completely blocks the blood flow to the target location. A balloon drive may be employed to control the inflation and deflation of the balloon. The balloon drive may subsequently be put under control of a computerized device.

The therapeutic agent may be any therapeutic agent suitable for the treatment of the pathological condition in the target tissue, such as an agent for treating brain-related disorders, a chemotherapeutic agent, and a gene-therapy agent. Preferably, the therapeutic agent is delivered to a target location, which is the artery in or near brain, a tumor, or a tissue in need of gene-therapy. In one embodiment, the therapeutic agent is delivered to the target location in bolus or in one or more pulses.

The present invention also teaches a method for the treatment of a pathological disorder in a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating a therapeutic agent into the drug delivery system; (3) occluding blood flow to the target location by inflating the balloon; (4) delivering the therapeutic agent to the target location; and (5) deflating the balloon after a short period of time.

### EXAMPLES

The following examples illustrate the present invention, which are set forth to aid in the understanding of the invention, and should not be construed to limit in any way the scope of the invention as defined in the claims which follow thereafter.

### Example 1

### Materials and Methods

After the approval of the protocol by the institution's animal care and use committee, the study was conducted on New Zealand White rabbits (1.5-2.0 kg. in weight). The animals were given full access to food and water prior to the experiment. The animals were sedated with an intramuscular ketamine (50 mg/kg). Intravenous access was obtained through an earlobe vein. Hydrocortisone 10 mg was given after the placement of an intravenous line, as it prevents hypotension, which sometimes occurs after surgical intervention in this animal species. Subsequently, the animal received 0.2 ml boluses of intravenous propofol (Diprivan® 1%, Astra Zeneca Pharmaceutical LP, Wilmington, Del.) as needed for maintaining adequate depth of anesthesia prior to tracheostomy. After infiltration of the incision site with local anesthetic, 0.25% bupivacaine with 1:200,000 epinephrine, a tracheotomy was undertaken for placement of endotracheal tube for mechanical ventilation by a Harvard small animal ventilator (Harvard Apparatus Inc., South Natick, Mass.). End-tidal CO₂ (ETCO₂) was continuously monitored with Novametrix Capnomac monitor (Novametrix Medical Systems Inc., Wallingford, Conn.). After securing the airway, anesthesia was maintained with intravenous infusion of propofol 1-2 ml/kg/hr, fentanyl 1-2 μg/kg/hr and vecuronium bromide 10-20 μg/kg/hr. A femoral arterial line was placed for monitoring mean arterial blood pressure (MAP).

The right common carotid artery was dissected in the neck and cannulated using a 20 cm-long PE-50 tubing (Becton Dickinson and Co., Spark, Md.). Correct identification of the internal carotid artery and its isolation was confirmed by the retinal discoloration test (Joshi et al., Retinal Discoloration Test. J. Cerebral Blood Flow Metabolism 24:305-3082004, 2004). Briefly, this test entails injection of 0.1-0.2 ml of 0.05% indigocarmine-blue, which changes the retinal reflex from red to blue when the internal carotid artery is correctly identified.

An esophageal temperature probe was used to monitor core temperature (e.g., Nova Therm, Novamed Inc., Rye, N.Y., or Mon-a-therm, 400H, Mallinckrodt Anesthesia Products, St. Louis, Mo.). The animal's temperature was kept constant between 37±1.0° C. using an electrically heated blanket. An intravenous infusion of fluid was given at 10 ml/kg/hr through an IVAC pump (IVAC 599 volumetric pump, IVAC Co., San Diego, Calif.). The intravenous infusion consisted of three fluids: ringer lactate, 5% dextrose, and 5% albumin mixed in a ratio of 3:1:1, respectively. Electroencephalographic recording (EEG), MAP, ETCO₂ and laser Doppler flows were continuously recorded on a computer using Powerlab software (AD Instruments Inc., Grand Junction, Colo.).

To measure cerebral blood flow (CBF), two laser Doppler probes (Probe# 407-1, Perimed Inc. Jarfalla, Sweden) were placed on either hemisphere. For probe placement, the animals were turned prone and positioned on a stereotactic frame. The skull was exposed through a midline incision. A 5×4 mm area of the skull was shaved with a drill, slightly anterior to the bregma and 1 mm lateral to the mid-line. The skull was shaved to expose the inner table, such that the cortical vessels could be seen through a fine layer of bone as described in literature (Morita-Tsuzuki, et al., Vasomotion in the rat cerebral microcirculation recorded by laser-Doppler flowmetry. Acta Physiologica Scandinavica 146:431-9, 1992). The probes were maneuvered to obtain a laser Doppler blood flow reading of 50-250 perfusion units (PU). Once the optimum site of placement was identified, the probes were secured within plastic retainers, and glued to the skull. Satisfactory probe placement was judged by an abrupt increase in probe reading during intracarotid injection of a small volume of saline (0.1 ml). Laser Doppler blood flow measurement technique provided a relative measure of blood flow changes in the tissue, therefore, laser Doppler blood flow values were normalized to the baseline value and were expressed as %-change from baseline value.

Fronto-parietal leads were placed and used to monitor the bilateral electrocerebral activity. Electrocerebral activity was monitored using standard stainless steel needle electrodes (impedance is <10 k Ohms). The frontal and the parietal needle electrodes were secured to the skull by small stainless steel screws. The neutral electrode placed in the temporalis muscle. Fronto-parietal electro-encephalographic signals were recorded using bioamplifier (ML136, AD Instruments, Grand Junction, Colo.), with a range of 100 mV, and electrocerebral activity (or electroencephalogram) recording mode having a pass-band 0.3 to 60 Hz. Analog data was sampled at 100 Hz per channel with an analog to digital converter, and displayed using the Chart 4.0 program (AD Instruments, Grand Junction, Colo.).

Electrocerebral silence was defined operationally, using a reference recording obtained with an identical recording technique from a known brain dead preparation following administration in intravenous KCl (Illievich, et al., Effects of hypothermia or anesthetics on hippocampal glutamate and glycine concentrations after repeated transient global cerebral ischemia. Anesthesiol. 80:177-86, 1994). A burst suppression pattern was evident during recovery from electrocerebral silence that was characterized by transient bursts of electrocerebral (or EEG) activity in the 30-50 μV range spaced with intervening period of electrocerebral silence. Electrocerebral recovery was defined as the return of electrocerebral activity with amplitudes and frequency composition comparable to baseline as judged by visual inspection (La Marca, et al., Cognitive and EEG recovery following bolus intravenous administration of anesthetic agents. Psychopharmacol. (Berl) 120:426-32, 1995). Total recovery time was defined as time between onset of electrocerebral silence after last injection and upon electrocerebral recovery. Post-drug silence time was the duration of time between the injection of last bolus to the return of detected EEG activity, generally a burst suppression pattern. Post-silence recovery time was described as the time between the onset of burst suppression to the return of EEG morphology comparable to the normal. Hemodynamic and cerebral blood flow parameters for each drug challenge were evaluated at three stages of the experiment: (i) at baseline; (ii) EEG silence with propofol boluses; (iii) electrocerebral recovery.

### Example 3

### Transient Flow Arrest Profoundly Increases the Duration of Electrocerebral Silence by Intracarotid Pentothal

For the present study, total recovery time was defined as time between the onset of electrocerebral silence after pentothal injection to electrocerebral activity comparable to baseline. Silence duration was the time elapsed between the injection of last bolus to the return of detectable electrocerebral activity, generally a burst-suppression pattern. Post-silence recovery time was described as the time between the onset of burst suppression to the return of electrocerebral activity comparable to the baseline. Hemodynamic and cerebral blood flow parameters for each drug were evaluated at three points of time: (i) baseline; (ii) during electrocerebral silence; and (iii) after recovery of electrocerebral activity.

Preliminary studies were undertaken to assess the optimum doses and cerebrovascular effects of drugs required to produce TCA. The preparation proved to be very tolerant to the effects of intravenous adenosine. Therefore, the inventor used an intravenous combination of esmolol 10 mg and 30 mg of adenosine, to produce severe systemic hypotension and flow arrest. This combination of drugs decreases the heart rate by 50-60%, and MAP and the laser Doppler flows to 20-30% of baseline values. Such a reduction in flow is sufficient to meet the criteria of flow arrest with laser Doppler measurements (Schmid-Elsaesser, et al., A critical reevaluation of the intraluminal thread model of focal cerebral ischemia: evidence of inadvertent premature reperfusion and subarachnoid hemorrhage in rats by laser-Doppler flowmetry. Stroke 29:2162-70, 1998).

The definitive study required comparisons between the effects of intracarotid pentothal with normal CBF and during flow arrest in the brain. There was a possibility that severe hypotension with the concurrent use of intra-arterial pentothal could injure the preparation. Due to the possibility of injury the inventor did not randomize the two interventions, but assessed the effects of pentothal before and after the hypotensive challenge. This helped assess the time-dependent, post-arrest, and residual drug effects on the preparation.

After baseline measurements of physiological parameters under normocapnic conditions were obtained, the animal received a standard injection of 3 mg of intracarotid pentothal. The loading dose of 1% pentothal is about 0.3±0.1 ml (Joshi, et al., Electrocerebral silence by intracarotid anesthetics does not affect early hyperemia after transient cerebral ischemia in rabbits. Anesth. Analg. 98:1454-9, 2004). Thus, a volume dose of 0.5 ml of 1% pentothal, assures adequate drug delivery to the brain to illicit consistent drug effects. Considering that 0.2 ml will remain in the dead space of the catheter and the stopcock, an effective dose of 3 mg was actually delivered to the cerebral circulation. Systemic hemodynamic effects, cerebrovascular, and the electrocerebral activity effects of the drugs were continuously recorded. The preparation was allowed to recover for 45 min. In the next stage, intravenous esmolol and adenosine were injected intravenously while pentothal was injected through the carotid artery. Electro-physiological and hemodynamic parameters were assessed thereafter. The preparation was then allowed to recover for another 45 min. After this, a repeat bolus of pentothal was injected via the intracarotid route.

The data is presented as mean±standard deviation. The hemodynamic and laser Doppler flow data recorded at three time points (baseline, silence and recovery) were normalized to baseline value. A P value of <0.05 was considered significant between the three challenges (pentothal-1, pentothal+arrest, and pentothal-2, ANOVA factorial). A P<0.0167 was considered significant between the three stages of each challenge (baseline, drug and recovery). All of which were evaluated by ANOVA repeated measures with Bonferroni Dunn test for multiple comparisons.

Discussed below are results obtained by the inventor in connection with the experiments of Examples 1 and 2:

Preliminary studies evaluated the effects of severe hypotension by esmolol and adenosine on electrophysiological and hemodynamic parameters. The preliminary studies were conducted on 4 animals to evaluate the cerebrovascular and electrophysiological effects of severe systemic hypotension in the absence of intracarotid drugs. As shown in Table I, injection of adenosine 30 mg and esmolol 10 mg decreased the MAP to 94±11 to 26±2 mm Hg, P<0.0001. During hypotension, the heart rate decreased from 257±20 to 132±26 beats/min, n=4, P=0.0003. The electrocerebral activity was attenuated during hypotension in all four animals immediately after injection of esmolol and adenosine. Blood flow declined from 147±78 to 47±29 PU, P=0.0083, i.e., to 20-30% of baseline values during hypotension. MAP and the HR returned to near baseline values within 3±1 minutes of drug injection. No inotropic support was required during recovery.

Definitive study was conducted on 10 animals. In one animal, the electrocerebral activity did not return to baseline amplitude and morphology after the arrest. Only data from the other nine animals were included in the final analysis. The definitive study involved three repeat challenges of drugs, (i) pentothal-1, (ii) pentothal+arrest, and (iii) pentothal-2, respectively. Intracarotid injection of pentothal prior to the flow arrest (pentothal-1) produced 45±5 seconds of electrocerebral silence (Table II). Post arrest injection of pentothal (pentothal-2) produced 67±27 seconds of electrocerebral silence that was not significantly different from pentothal-1 (n=9, P=0.132). The total recovery time was significantly prolonged during pentothal+arrest (291±60 seconds) but was comparable between pentothal-1 (126±29 seconds) and pentothal-2 (161±71 seconds). However, the time between the post-silence recovery was similar in the three groups pentothal-1, pentothal+arrest, and pentothal-2 (81±27, 85±27, and 94±55 seconds, respectively). Injection of pentothal 3 mg during flow arrest produced 206±46 seconds of silence that was significantly different from pentothal-1 (46±5 seconds, P<0.0001) and pentothal-2 (67±27 seconds, P<0.0001). The MAP, HR, ETC02 and laser Doppler flows were significantly lower during pentothal+arrest (Table III and IV). Ipsilateral laser Doppler flow were 130±59 to 33±11 P.U., i.e., to <20-30% of baseline values. Cerebral and systemic hemodynamic parameters were comparable between the two pentothal challenges.

Although TCA has been extensively used during endovascular surgery, this is the first study to evaluate the possibility of using flow arrest as a tool to enhance delivery of drugs to the brain. The inventor observed that intracarotid injection of pentothal during flow arrest, significantly prolonged the duration of electrocerebral silence, although post-silence recovery time was similar with all the three challenges. These results suggest that modulation of blood flow to the brain is a critical factor in influencing the efficacy of intra-arterial drugs. The data further suggest that the increase in duration of electrocerebral silence is due to higher concentrations of drug in the brain, and not due to slow rate of drug washout once flow is restored.

TABLE I
Preliminary Studies Showing The Effect Of Flow Arrest On
Hemodynamic And Cerebral Blood Flow Parameters

| n = 4 | Baseline | Arrest | Recovery |
|---|---|---|---|
| Heart Rate (bpm) | 257 ± 20 | 133 ± 26# | 235 ± 25 |
| Temperature (° C.) | 36.4 ± .7 | 36.3 ± .7 | 36.4 ± .6 |
| ETCO₂ (mm Hg) | 38 ± 2 | 31 ± 7# | 37 ± 3 |
| Respiratory rate | 32 ± 3 | 32 ± 3 | 32 ± 3 |
| (br. pm) | | | |
| MAP (mm Hg) | 94 ± 11 | 27 ± 2# | 97 ± 8 |
| ILD Flow (PU) | 147.14 ± 78.05 | 46.84 ± 28.75# | 170.34 ± 92.24 |
| CLD Flow (PU) | 118.67 ± 58.65 | 51.51 ± 28.81# | 124.88 ± 61.19 |
| % Δ-ILD | 100 | 31.03 ± 5.64# | 114.23 ± 10.86 |
| % Δ-CLD | 100 | 41.64 ± 5.76# | 105.89 ± 10.42 |

Abbreviations:
bpm: beats per minute,
ETCO₂: End-tidal carbon dioxide concentration,
br. pm: breaths per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
#significant post-hoc differences between stages (P < 0.0167).

TABLE II
The Effect of Intracarotid Pentothal
on the Duration of EEG Parameters

| N = 9 | Pentothal-1 | Pentothal + Arrest | Pentothal-2 |
|---|---|---|---|
| Silence Duration(s) | 45 ± 5 | 206 ± 46* | 67 ± 27 |
| Total Recovery | 126 ± 29 | 291 ± 60* | 161 ± 71 |
| Time(s) | | | |
| Post-silence recovery | 81 ± 27 | 85 ± 27 | 94 ± 55 |
| time (s) | | | |

*significant differences between challenges (P < 0.05)

Adenosine and esmolol are both exceedingly short acting drugs. A combination of these drugs was sufficient to produce a severe reduction in laser Doppler flow to 20-30% of baseline values, which is sufficient to meet the criteria of flow arrest by laser Doppler measurements. However, the use of such high doses of the drug made randomization difficult. Rather than randomize the drugs, the inventor tested the response to pentothal before and after the pharmacological flow arrest. By using two control challenges the inventor could assess changes in the preparation due to time, possible ischemic injury and the residual effects of systemic drugs. The results of pentothal-1 and pentothal-2 challenges were fairly similar, which suggest a minimal residual effect of flow arrest on electrocerebral response to intracarotid pentothal.

TABLE III
Changes In Non-Hemodynamic Parameters
During The Three Pentothal Challenges

| N = 9 | | Baseline | Drug | Recovery |
|---|---|---|---|---|
| Temperature | Pentothal-1 | 36.6 ± 1.0 | 36.5 ± 1.0 | 36.5 ± 1.0 |
| (° C.) | Pentothal + Arrest | 36.4 ± 0.9 | 36.4 ± 0.8 | 36.3 ± 0.8 |
| | Pentothal-2 | 36.4 ± 0.8 | 36.5 ± 0.7 | 36.4 ± 0.7 |
| Respiratory | Pentothal-1 | 30 ± 5 | 30 ± 5 | 31 ± 5 |
| rate (br. pm) | Pentothal + Arrest | 31 ± 4 | 31 ± 4 | 31 ± 4 |
| | Pentothal-2 | 31 ± 4 | 31 ± 4 | 31 ± 4 |
| ETCO₂ | Pentothal-1 | 37 ± 2 | 37 ± 2* | 36 ± 2 |
| (mm Hg) | Pentothal + Arrest | 37 ± 3 | 31 ± 3# | 34 ± 4 |
| | Pentothal-2 | 37 ± 3 | 37 ± 4* | 36 ± 4 |

Abbreviations:
ETCO₂: End-tidal carbon dioxide concentration,
br. pm: breaths per minute.
*significant post hoc differences between challenges (P < 0.05),
#significant post-hoc differences between stages (P < 0.0167).

TABLE IV
Changes In Systemic And Cerebrovascular Hemodynamic
Parameters During Intracarotid Injection Of Pentothal

| N = 9 | Challenge | Baseline | Drug | Recovery |
|---|---|---|---|---|
| Heart Rate | Pentothal-1 | 262 ± 42 | 262 ± 36* | 241 ± 29 |
| (bpm) | Pentothal + Arrest | 265 ± 21 | 125 ± 19# | 258 ± 44 |
| | Pentothal-2 | 261 ± 23 | 254 ± 20* | 268 ± 22 |
| MAP (mm Hg) | Pentothal-1 | 101 ± 14 | 94 ± 17* | 102 ± 7 |
| | Pentothal + Arrest | 98 ± 11 | 26 ± 2# | 91 ± 14 |
| | Pentothal-2 | 96 ± 13 | 91 ± 21* | 99 ± 11 |
| ILD Flow (PU) | Pentothal-1 | 141 ± 73 | 147 ± 83* | 140 ± 66 |
| | Pentothal + Arrest | 130 ± 59 | 33 ± 11# | 161 ± 81 |
| | Pentothal-2 | 143 ± 76 | 140 ± 55* | 129 ± 68 |
| CLD Flow | Pentothal-1 | 106 ± 45 | 111 ± 50* | 93 ± 23 |
| (PU) | Pentothal + Arrest | 114 ± 48 | 41 ± 21# | 106 ± 34 |
| | Pentothal-2 | 111 ± 53 | 118 ± 41* | 89 ± 38 |
| % Δ-ILD | Pentothal-1 | 100 ± 0 | 106 ± 51* | 101 ± 29 |
| | Pentothal + Arrest | 100 ± 0 | 27 ± 8# | 124 ± 33 |
| | Pentothal-2 | 100 ± 0 | 104 ± 30* | 92 ± 38 |
| % Δ-CLD | Pentothal-1 | 100 ± 0 | 106 ± 38* | 94 ± 19 |
| | Pentothal + Arrest | 100 ± 0 | 36 ± 9# | 100 ± 32 |
| | Pentothal-2 | 100 ± 0 | 112 ± 26* | 83 ± 22 |

Abbreviations:
bpm: beats per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
*significant post hoc differences between challenges (P < 0.05),
#significant post-hoc differences between stages (P < 0.0167).

In clinical practice, intra-arterial drugs have been used effectively during the treatment of cerebral vasospasm, a condition of low cerebral blood flow (Oskouian, et al., Multimodal quantitation of the effects of endovascular therapy for vasospasm on cerebral blood flow, transcranial doppler ultrasonographic velocities, and cerebral artery diameters. Neurosurgery 51:30-41, 2002). However, intra-arterial delivery has been less efficacious in other settings, such as in the treatment of brain tumors (Oldfield, et al., Reduced systemic drug exposure by combining intra-arterial chemotherapy with hemoperfusion of regional venous drainage. J Neurosurg. 63:726-32, 1985). A number of factors, such as inadequate penetration of blood brain barrier by drugs, may explain the therapeutic failures of intra-arterial chemotherapy. However, no attempt was made in the past to modulate blood flow to enhance intra-arterial drug delivery to the brain, which is a key determinant of drug delivery to the brain. By using computer simulations, Dedrick R. L. reported that intra-arterial drugs was efficacious in three specific situations: drugs with high systemic clearance, drugs with selective brain uptake, and drugs administered in areas of low regional blood flow (Dedrick R. L., Arterial drug infusion: pharmacokinetic problems and pitfalls. Journal of the National Cancer Institute 80:84-9, 1988). It is to be noted that when anesthetic drugs are administered intravenously, augmentation of CBF enhances drug delivery to the brain (Upton, et al., The effect of altered cerebral blood flow on the cerebral kinetics of thiopental and propofol in sheep. Anesthesiology 93:1085-94, 2000). The converse seems to be true with intra-arterial drug delivery.

There are two outstanding concerns in employing flow arrest to the brain. The first concern is the possibility of ischemic cerebral injury and the second concern is the occurrence reactive hyperemia. In the present model, the duration of flow arrest was very transient <20 seconds and the flows rapidly returned to near baseline values within 1 min of hypotension. The inventor observed transient attenuation of electrocerebral activity during flow arrest that in the absence of pentothal, and the electrocerebral activity rapidly (<45 seconds) returned to baseline amplitude and morphology. These data suggest that the magnitude of reduction of flow in the present model was not associated with injury. If flow arrest is clinically used, the duration of flow arrest has to be sufficiently short so as to avoid any ischemic injury. The second hazard of flow arrest is the reactive hyperemia. While the clinical impact of post-ischemic reactive hyperemia can be debated, such an increase in flow will enhance drug elimination from the brain. The inventor did not observe a significant increase in laser Doppler flow after transient flow arrest during the preliminary studies. Previously, the inventor has observed significant increases in laser Doppler flows occur in the experimental model when ischemia last for about 10 min (Joshi, et al., Electrocerebral silence by intracarotid anesthetics does not affect early hyperemia after transient cerebral ischemia in rabbits. Anesth. Analg. 98:1454-9, 2004). It seems that transient ischemia of <20 seconds duration does not result in significant hyperemia.

There are few studies that have assessed electrocerebral activity changes as a function of the concentrations of pentothal in the brain. In sheep, electrocerebral silence is evident when tissue concentration of pentothal that produce electrocerebral silence is about 0.50 mg/dl (Mather, et al., Electroencephalographic effects of thiopentone and its enantiomers in the rat. Life Sciences 66:105-14, 2000). However, there are a number of studies that correlate electro-encephalographic changes and arterial pentothal concentrations. In the present study, the administration of pentothal during flow arrest prolonged the duration of electrocerebral silence, however, the recovery of electroencephalographic morphology after the end of silence was not affected by flow arrest. Recovery from electrocerebral silence will be a function of peak tissue concentrations, redistributive half-life of pentothal, and the regional blood flow. Relative to the prolongation in the duration of electrocerebral silence (3-5 folds) with pentothal+arrest vs. pentothal-1 and 2, the ipsilateral CBF remained low during recovery, and was comparable with the three challenges. Thus, the decrease in blood flow could not have explained the increased duration of electrocerebral silence. The results of the present study suggest that the prolongation of electrocerebral silence by intracarotid pentothal during flow arrest, was primarily due to a higher tissue concentrations.

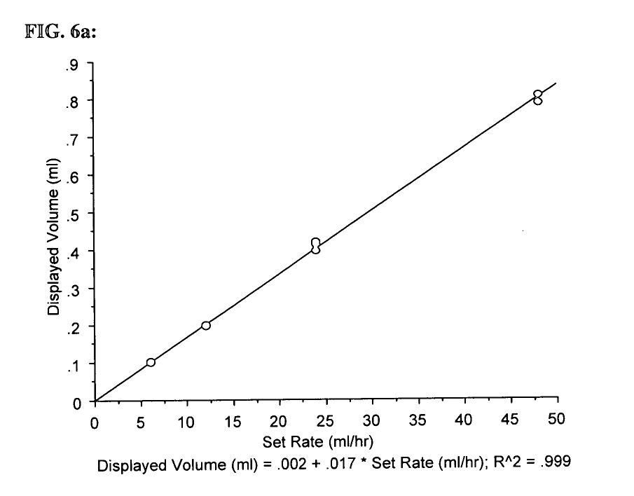

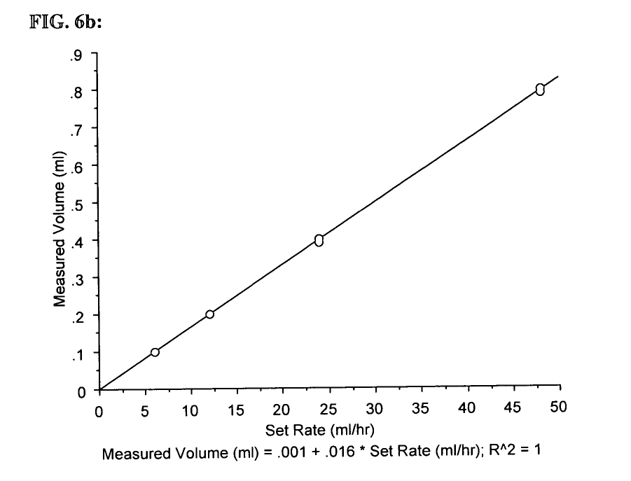

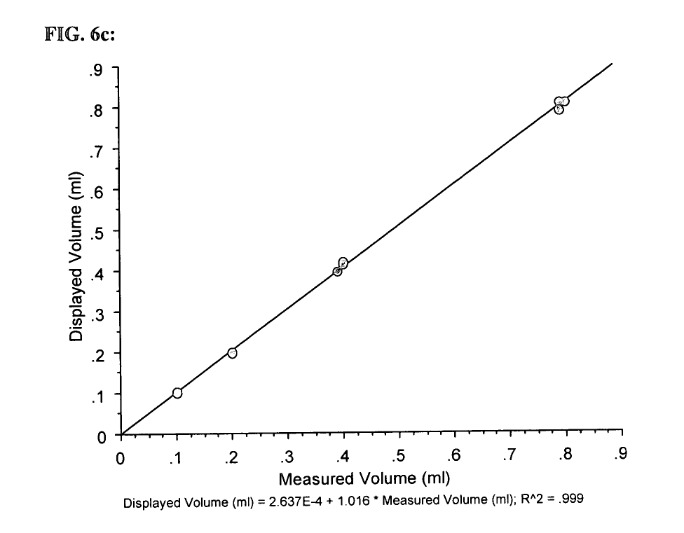

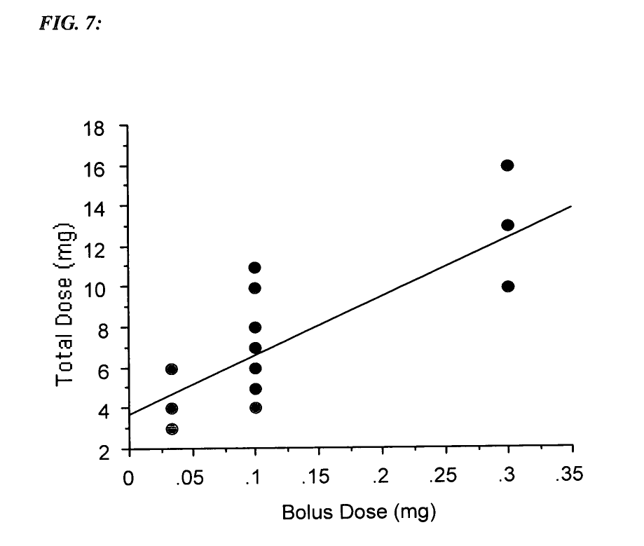

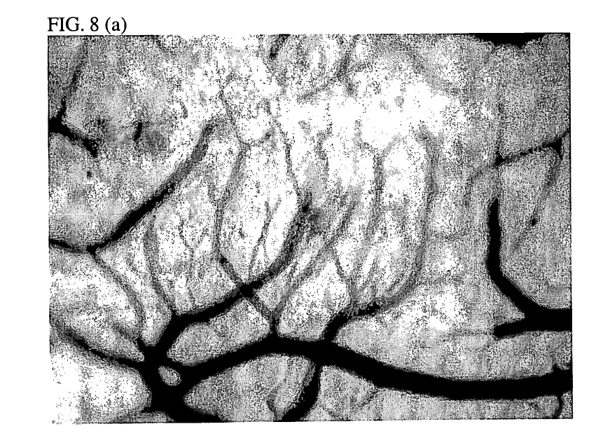

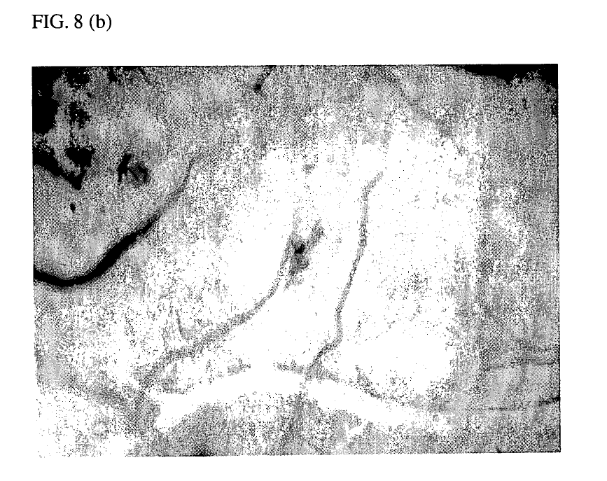

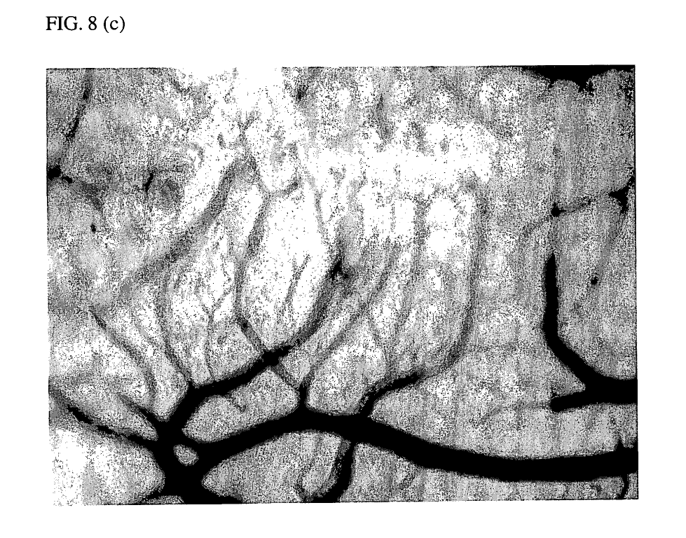

FIGS. 6-8 shows passage delivery of pure drug with bolus injection which also shows that bolus delivery momentarily overwhelms blood flow to the brain. Thus, pure drug reaches the brain tissue. In this respect, the drug is free of any protein binding and achieves high tissue concentrations.

The present study demonstrates that the administration of intracarotid pentothal during flow arrest increases the duration of drug effect, which indicates that modulation of blood flow might be an important tool in enhancing intra-arterial drug delivery to the brain.

While the invention has been described and illustrated in connection with preferred embodiments, many variations and modifications, as will be evident to those skilled in the art, may be made without departing from the spirit and scope of the invention. The invention is thus not limited to the precise details of construction set forth above as such variations and modifications are intended to be included within the spirit and scope of the invention.

What is claimed:

1. A method of infusing a fluid into a blood vessel comprising:
reducing blood flow in the blood vessel; and
administering the fluid into the blood vessel in programmed pulses defined by programmed pulse variables comprising a fluid flow rate, a frequency, and a duration.

2. The method of claim 1, wherein blood flow is reduced for less than about 20 seconds.

3. The method of claim 1, wherein blood flow is reduced from about 0% to about 50% of a baseline blood flow.

4. The method of claim 1, wherein blood flow is reduced with at least one occlusion catheter.

5. The method of claim 1, wherein fluid is administered with an infusion catheter.

6. The method of claim 1, wherein blood flow is reduced and fluid is administered with a multiple lumen catheter comprising at least one first lumen for infusing fluid into the blood vessel and at least one second lumen for reducing blood flow in the blood vessel.

7. The method of claim 6, wherein the multiple lumen catheter is a side-by-side catheter.

8. The method of claim 6, wherein the multiple lumens of the multiple lumen catheter are disposed in a co-axial arrangement.

9. The method of claim 1, comprising administering the fluid at a fluid flow rate of about 2% to about 5% of a baseline arterial blood flow of a carotid artery.

10. The method of claim 1, comprising administering the fluid in programmed pulses at the duration of about one to five seconds.

11. A programmed pulse infusion device comprising:
a pump;
a controller with associated memory interfacing with the pump;
a fluid reservoir containing a fluid feeding the pump, the controller providing a drive signal for the pump to deliver the fluid for infusion into a blood vessel in programmed pulses defined by programmed pulse variables comprising a fluid flow rate, a frequency, and a duration; and
a balloon drive for operating an occlusion catheter for reducing blood flow in the blood vessel.

12. The device of claim 11, wherein blood flow is reduced for less than about 20 seconds.

13. The device of claim 11, wherein blood flow is reduced from about 0% to about 50% of a baseline blood flow.

14. The device of claim 11, wherein blood flow is reduced with at least one occlusion catheter.

15. The device of claim 11, wherein fluid is administered with an infusion catheter.

16. The device of claim 11, wherein blood flow is reduced and fluid is administered with a multiple lumen catheter comprising at least one first lumen for infusing fluid into the blood vessel and at least one second lumen for reducing blood flow in the blood vessel.

17. The device of claim 16, wherein the multiple lumen catheter is a side-by-side catheter.

18. The device of claim 16, wherein the multiple lumens of the multiple lumen catheter are disposed in a co-axial arrangement.

19. The method of claim 11, comprising administering the fluid at a fluid flow rate of about 2% to about 5% of a baseline arterial blood flow of a carotid artery.

20. A computer readable medium comprising program code that when executed enables a user to determine values of programmed pulse variables for infusing a fluid in a blood vessel in programmed pulses defined by the programmed pulse variables comprising a fluid flow rate, a frequency, and a duration, while blood flow is reduced in the blood vessel.

21. The computer readable medium of claim 20, wherein blood flow is reduced for less than about 20 seconds.

22. The computer readable medium of claim 20, wherein blood flow is reduced from about 0% to about 50% of a baseline blood flow.

23. The computer readable medium of claim 20, wherein blood flow is reduced with at least one occlusion catheter.

24. The computer readable medium of claim 20, wherein fluid is administered with an infusion catheter.

25. The computer readable medium of claim 20, wherein blood flow is reduced and fluid is administered with a multiple lumen catheter comprising at least one first lumen for infusing fluid into the blood vessel and at least one second lumen for reducing blood flow in the blood vessel.

26. The computer readable medium of claim 25, wherein the multiple lumen catheter is a side-by-side catheter.

27. The computer readable medium of claim 25, wherein the multiple lumens of the multiple lumen catheter are disposed in a co-axial arrangement.

28. The computer readable medium of claim 20, comprising administering the fluid at a fluid flow rate of about 2% to about 5% of a baseline arterial blood flow of a carotid artery.
